(19) United States
(12) Patent Application Publication
(10) Pub. No.: US 2016/0066874 A1
(43) Pub. Date: Mar. 10, 2016

(54) ATTENUATION CORRECTION OF POSITRON EMISSION TOMOGRAPHY DATA USING MAGNETIC RESONANCE IMAGES DEPICTING BONE DENSITY VARIATIONS

(71) Applicant: THE GENERAL HOSPITAL CORPORATION, BOSTON, MA (US)

(72) Inventors: CHUAN HUANG, BOSTON, MA (US); JEROME L. ACKERMAN, NEWTON, MA (US); JINSONG OUYANG, LEXINGTON, MA (US)

(21) Appl. No.: 14/850,175

(22) Filed: Sep. 10, 2015

Related U.S. Application Data
(60) Provisional application No. 62/048,521, filed on Sep. 10, 2014.

(51) Int. Cl. A61B 6/00; G01R 33/48; A61B 6/03

(52) U.S. Cl. CPC A61B 6/5258; A61B 6/037; A61B 6/582; A61B 6/469; A61B 6/505; G01R 33/481; G01R 33/4828

(57) ABSTRACT

Systems and methods for performing attenuation correction on positron emission tomography (“PET”) data using images acquired with a magnetic resonance imaging (“MRI”) system are provided. Preferably, the magnetic resonance images are acquired using a pulse sequence that produces magnetic resonance signals from bone tissue that can be distinguished by variations in bone density. Images acquired in this manner can provide information about intra-subject and inter-subject variations in bone density, thereby resulting in more accurate attenuation correction in bone tissues.

### CROSS-REFERENCE TO RELATED APPLICATIONS

This application claims the benefit of U.S. Provisional Patent Application Ser. No. 62/048,521, filed on Sep. 10, 2014, and entitled “PET Attenuation Correction for PET-MR.”

### STATEMENT REGARDING FEDERALLY SPONSORED RESEARCH

This invention was made with government support under MH106994, EB012326, CA165221, HL110241, HL118261 and EB015896 awarded by the National Institutes of Health. The government has certain rights in the invention.

### BACKGROUND OF THE INVENTION

The field of the invention is systems and methods for positron emission tomography. More particularly, the invention relates to systems and methods for attenuation correction of data acquired with positron emission tomography.

In recent years, combined positron emission tomography (“PET”) and magnetic resonance imaging (“MRI) has generated significant interest. The integration of these two medical imaging modalities with very different physics allows for the development of many novel, synergistic techniques and applications. One of the biggest hurdles of quantitative PET-MR, however, is the need for accurate PET attenuation correction (AC), especially for bone.

In stand-alone PET, attenuation maps (μ-maps) are obtained by a separate transmission scan using an external source. In combined PET-CT systems, the attenuation coefficients are measured with x-rays (with energies often in the neighborhood of 100 keV) and are remapped to estimate the attenuation coefficients for the 511 keV photons encountered in PET. For combined PET-MR, it is desirable to derive the PET μ-map from magnetic resonance images so the subject being imaged does not need to be exposed to unnecessary doses of radiation. The problem with basing attenuation correction on magnetic resonance images, however, is that the attenuation coefficient of the photons depends on the electron density, but clinical magnetic resonance image contrast arises from proton spin density and spin-spin (i.e., T₂) and spin-lattice (e.g., T₁) relaxation.

The standard approach for MRI-based PET attenuation correction is to segment a magnetic resonance image volume into different tissue classes and then assign the corresponding attenuation coefficients to the segmented tissue classes to create a μ-map. Segmentation techniques that have been previously proposed include atlas-based techniques and those based on ultrashort echo time (“UTE”) or zero echo time (“ZTE”) pulse sequences. Although atlas-based μ-maps can provide continuous bone attenuation, these approaches are generally not feasible in practice because anatomy-based registrations are extremely challenging. Moreover, atlas-based approaches do not capture inter-subject bone density variations.

UTE-based approaches can identify bones in the body, but cannot be used to measure bone density variation, which can be between 700 Hounsfield units (HU) for cancellous bone to 3000 HU for dense bone. Without capturing the intra-subject and inter-subject bone density variation, a UTE-based μ-map will inevitably lead to bias in reconstructed PET images.

Thus, there remains a need to provide MRI-based attenuation correction for PET data, especially for attenuation correction in bone tissue that accounts for intra-subject and inter-subject bone density variations.

### SUMMARY OF THE INVENTION

The present invention overcomes the aforementioned drawbacks by providing a method for correcting positron emission tomography (“PET”) data for photon attenuation effects using magnetic resonance imaging (“MRI”). A magnetic resonance image that contains data about bone density variations in a subject is provided to a computer system. Linear photon attenuation coefficients are computed with the computer system by mapping signal intensity values in the magnetic resonance image to the linear photon attenuation coefficients. PET data acquired from the subject are then provided to the computer system for photon attenuation correction. Photon attenuation corrected PET data are computed with the computer system by correcting the provided PET data using the linear photon attenuation coefficients.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an example pulse sequence diagram for a water- and fat-suppressed projection imaging (“WASPI”) pulse sequence;

FIG. 2 is a flowchart setting forth the steps of an example method for attenuation correcting PET data using magnetic resonance images that depict bone density variations in a subject;

FIG. 3 is block diagram of an example of a PET system that can be configured as a stand-alone PET system or as part of an integrated PET-MR system; and

FIG. 4 is a block diagram of an example MRI system that can be configured as a stand-alone MRI system or as part of an integrated PET-MR system.

### DETAILED DESCRIPTION OF THE INVENTION

Described here are systems and methods for performing attenuation correction on positron emission tomography (“PET”) data using images acquired with a magnetic resonance imaging (“MRI”) system. Preferably, the magnetic resonance images are acquired using a pulse sequence that produces magnetic resonance signals from bone tissue that can be distinguished by variations in bone density. For instance, the images can be acquired using a water- and fat-suppressed projection imaging (“WASPI”) pulse sequence. Images acquired in this manner can provide information about intra-subject and inter-subject variations in bone density, thereby resulting in more accurate attenuation correction in bone tissues.

An example of the WASPI pulse sequence is described by Y. Wu, et al., in “Density of organic matrix of native mineralized bone measured by water-and fat-suppressed proton projection MRI,” Magnetic Resonance in Medicine, 2003; 50:59-68. In general, the WASPI pulse sequence is a three-dimensional radial zero echo time (“TE”) pulse sequence with fat and water suppression. The magnetic resonance signals acquired with this pulse sequence generally include signals only from very short T₂ protons, such as those associated with immobile proteins and tightly bound water in the bone matrix. Signal intensity values in these images are proportional to bone matrix density, which for normally mineralized bone is in turn proportional to bone mineral density.

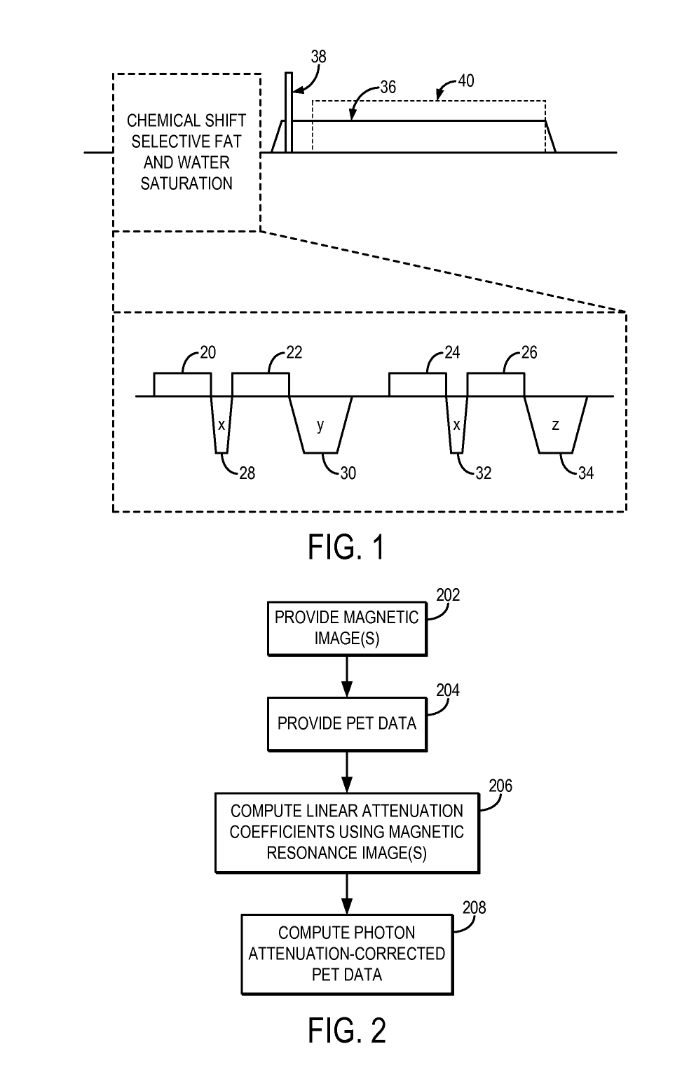

As shown in FIG. 1, the WASPI pulse sequence first saturates the fluid (molecularly mobile) tissue constituents with chemical shift selective radio frequency (“RF”) pulses 20, 22 at the water frequency and with chemical shift selective RF pulses 24, 26 at the fat frequencies. Each saturation pulse (20, 22, 24, 26) is followed by a crusher gradient pulse 28, 30, 32, 34 to dephase the fluid signals. A fixed-amplitude gradient 36 is then turned on, and a very brief (e.g., 10 μs) rectangular hard RF pulse 38 covering the full bandwidth of the field-of-view is applied to elicit a free induction decay (“FID”) signal that is sampled to yield a single radial line in k-space. The direction of the fixed-amplitude gradient 36 is advanced to successive orientations to cover a spherical volume of k-space. The acquired data can be reconstructed using a regridding algorithm or a suitable iterative reconstruction algorithm.

The chemical shift selective RF pulses (20, 22, 24, 26) generally include four 90 degree RF pulses. The timing and gradient moments of the dephasing gradients (28, 30, 32, 34) are generally chosen to avoid echo formation. Preferably, receiver dead time between the hard RF pulse 38 and the data acquisition window 40 is minimized in order to accurately acquire the center of k-space, which is important for obtaining a quantitatively accurate measurement of bone density.

In some instances, the dead time can be minimized by using a fast switch from transmitting mode to receiving mode. As one example, fast switching can be achieved using a transmit/receive switch based on quadrature hybrids and standard silicon switching diodes (rather than PIN diodes), which may be capable of reducing dead time from 100-200 μs to about 10 μs.

To further improve the fidelity of k-space data near the origin, an additional acquisition of a small number of k-space radii can be implemented with a reduced gradient strength for the fixed strength gradient 36, which enables central k-space points to be acquired at times farther away from the RF pulse and its switching transients.

By eliminating echo formation and slice selection, and by minimizing time delays following the RF excitation pulse, the WASPI sequence enables a very high fidelity acquisition of very short-T₂ signals.

Referring now to FIG. 2, a flowchart setting forth the steps of an example of a method for correcting PET data for photon attenuation effects is illustrated. One advantage of this method is that it accounts for intra-subject and inter-subject bone attenuation variation that is not accounted for in other MRI-based methods for attenuation correcting PET data.

One or more images acquired with an MRI system are provided to a computer system, as indicated at step 202. Preferably, the one or more images are acquired using a pulse sequence that enables accurate distinctions of intra-subject and inter-subject bone density variations. As one example, the pulse sequence may be a WASPI pulse sequence, such as the one described above. In some embodiments, providing the one or more images acquired with the MRI system includes retrieving previously acquired images from a data storage device. In other embodiments, providing the one or more images includes acquiring data using an MRI system and reconstructing images therefrom. As one example, the one or more images can be acquired using an integrated PET-MRI system; however, the one or more images can also be acquired with a stand-alone MRI system.

PET data are also provided to the computer system, as indicated at step 204. In some embodiments, providing the PET data includes retrieving previously acquired PET data from a data storage device. In other embodiments, providing the PET data includes acquiring PET data using a PET system. The magnetic resonance images can be acquired substantially contemporaneously with the PET data using an integrated PET-MRI system, or can be acquired serially, whether using an integrated PET-MRI system or using stand-alone PET and MRI systems.

Linear photon attenuation coefficients are computed using the one or more magnetic resonance images, as indicated at step 206. In some embodiments, the photon attenuation coefficients can be computed by mapping the signal intensity values in the one or more magnetic resonance images to the linear photon attenuation coefficients using an appropriately calibrated mapping function. The mapping function can be generated by comparing the signal intensity values in the one or more magnetic resonance images with calibration data. Because of the non-quantitative nature of MRI, calibration is needed for converting the signal intensity values in the magnetic resonance images to bone density values for the purpose of attenuation correction.

As one example, the calibration data can include magnetic resonance signal intensity values indicative of a material with a known density. For instance, the calibration data can be determined from a separate magnetic resonance image depicting a calibration phantom with known materials with known densities. Calibration data can be determined from such a phantom image by associating the magnetic resonance signal intensity values for regions in the phantom image containing the known material with known density to those known density values.

As another example, the calibration data can be determined from a calibration phantom that is depicted in the one or more magnetic resonance images of the subject. For instance, a calibration phantom can be positioned proximate to the subject during imaging such that a separate data acquisition is not needed to provide the calibration data. A region-of-interest (“ROI”) containing the calibration phantom can be identified in the one or more magnetic resonance images and the signal intensity values in that ROI can be associated with the known density of the material in the calibration phantom.

A suitable calibration phantom can include pellets or other objects composed of a polymer blend with a known density. As one example, the polymer blend can be a 20 percent/80 percent blend of poly(ethylene oxide) and poly(methyl methacrylate) (PEO/PMMA).

In one non-limiting example, magnetic resonance signal intensities can be associated with bone matrix density values as follows. The WASPI-derived bone matrix density, D_{BmW}, and a calibration density value for an i^{th} region-of-interest in calibration data, D_{CWi}, can be calculated in units of MRI intensity per pixel:

$$\begin{matrix}{D}_{BmW} = \frac{{M}_{BmW}}{{V}_{Bt}} ; \\ and & ( 1 ) \\ {D}_{CWi} = \frac{{M}_{CWi}}{{V}_{Ci}} ; & ( 2 )\end{matrix}$$

where M_{BmW} is the mass of bone matrix expressed in terms of WASPI-signal intensity, which is the sum of WASPI signal intensities over all pixels within the bone tissue volume, V_{Bt}; V_{Bt} is the bone tissue volume, which is the total number of pixels of bone tissue in a non-suppressed magnetic resonance image; M_{CWi} is the mass of the calibration region, such as the mass of a given pellet or object in a calibration phantom, expressed in terms of WASPI image intensity, and which is the sum of WASPI intensities over all pixels within the volume of the calibration region, V_{Ci}; and V_{Ci} is the volume of a calibration region, which is the total number of pixels associated with the calibration region in each WASPI image.

The physical density, D_{CPi}, of the calibration region in units of g cm⁻³ is then computed by dividing the physical mass of the material in the calibration region by the physical volume of the calibration region. A calibration curve of D_{CWi} versus D_{CPi} can then be created using linear regression of the two sets of data for each WASPI image. The D_{BmW} values are then converted to physical density values, D_{BmP}, by using the formula found in the linear regression. In some instances, conversion factors can be used to convert the physical density values to matrix mass density or matrix protein density values. As an example, the conversion factors can be derived from gravimetric and amino acid analyses of bone tissue density of a bone tissue sample, which are then correlated with physical density values, D_{BmP}, of the same sample through linear regression.

In another example, however, the calibration data can be determined by identifying one or more ROIs in the magnetic resonance images of the subject, where the one or more ROIs correspond to one or more tissues having known densities. As one non-limiting example, two ROIs can be identified, one containing cortical bone, which is highly attenuating, and the other containing spongy bone, which is less attenuating than cortical bone.

In some other embodiments, the linear photon attenuation coefficients can be computed in step 206 based on quantification of immobile proteins in the bone matrix by calibrating proton signal intensity values using calibration data obtained without water and fat suppression. The bone density can be derived from this quantification of immobile proteins and the signal intensity values in the one or more magnetic resonance images.

After the linear attenuation coefficients are computed they are used to compute photon attenuation corrected PET data, as indicated at step 208. The corrected data can then be reconstructed to produce a PET image volume using any suitable reconstruction technique.

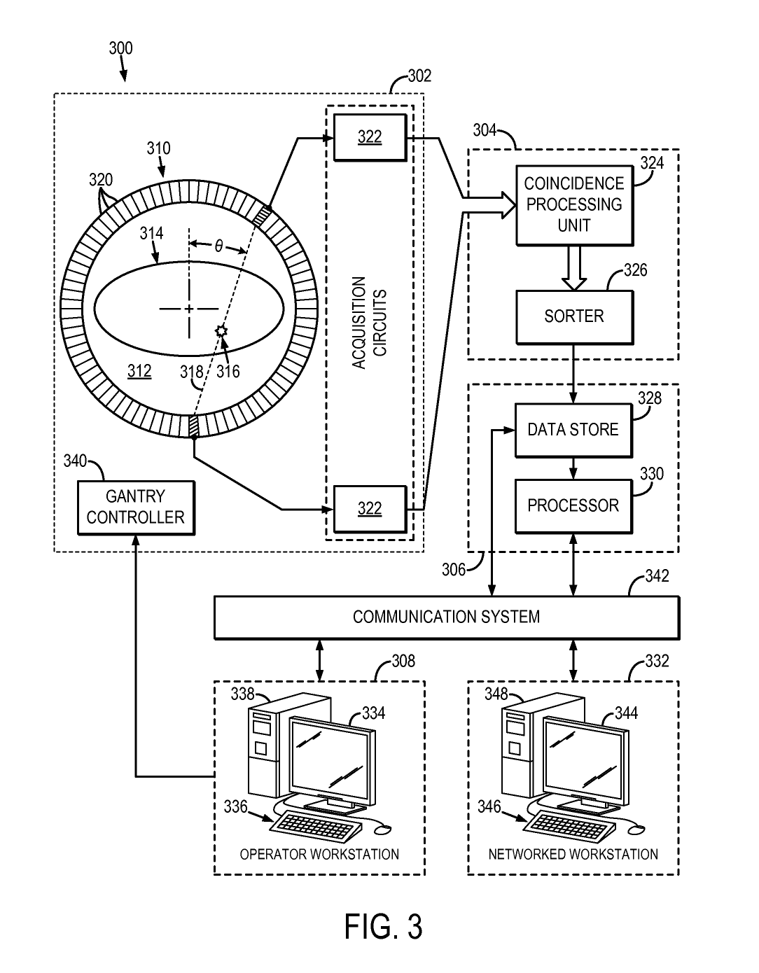

Referring now to FIG. 3, an example of a positron emission tomography (“PET”) system 300 is illustrated. The PET system 300 generally includes an imaging hardware system 302, a data acquisition system 304, a data processing system 306, and an operator workstation 308. In some embodiments, the PET system 300 corresponds to a stand-alone PET system; however, it will be appreciated by those skilled in the art that the PET system 300 can also be integrated in a combined imaging system, such as a combined PET and x-ray computed tomography (“CT”) system, or a combined PET and magnetic resonance imaging (“MRI”) system.

The imaging hardware system 302 generally includes a PET scanner having a radiation detector ring assembly 310 that is centered about the bore 312 of the PET scanner. The bore 312 of the PET scanner is sized to receive a subject 314 for examination. Prior to imaging, the subject 314 is administered a radioisotope, such as a radionuclide or radiotracer. Positrons are emitted by the radioisotope as it undergoes radioactive decay. These positrons travel a short distance before encountering electrons at which time the positron and electron annihilate. The positron-electron annihilation event 316 generates two photons that travel in opposite directions along a generally straight line 318.

The radiation detector ring assembly 310 is formed of multiple radiation detectors 320. By way of example, each radiation detector 320 may include one or more scintillators and one or more photo detectors. Examples of photo detectors that may be used in the radiation detectors 320 include photomultiplier tubes (“PMTs”), silicon photomultipliers (“SiPMs”), or avalanche photodiodes (“APDs”). The radiation detectors 320 are thus configured to produce a signal responsive to the photons generated by annihilation events 316. The signal responsive to the detection of a photon is communicated to a set of acquisition circuits 322. The acquisition circuits 322 receive the photon detection signals and produce signals that indicate the coordinates of each detected photon, the total energy associated with each detected photon, and the time at which each photon was detected. These data signals are sent the data acquisition system 304 where they are processed to identify detected photons that correspond to an annihilation event 316.

The data acquisition system 304 generally includes a coincidence processing unit 324 and a sorter 326. The coincidence processing unit 324 periodically samples the data signals produced by the acquisition circuits 322. The coincidence processing unit 324 assembles the information about each photon detection event into a set of numbers that indicate precisely when the event took place and the position in which the event was detected. This event data is then processed by the coincidence processing unit 324 to determine if any two detected photons correspond to a valid coincidence event.

The coincidence processing unit 324 determines if any two detected photons are in coincidence as follows. First, the times at which two photons were detected must be within a predetermined time window, for example, within 6-12 nanoseconds of each other. Second, the locations at which the two photons were detected must lie on a line 318 that passes through the field of view in the PET scanner bore 312. Each valid coincidence event represents the line 318 connecting the two radiation detectors 320 along which the annihilation event 316 occurred, which is referred to as a line-of-response (“LOR”). The data corresponding to each identified valid coincidence event is stored as coincidence data, which represents the near-simultaneous detection of photons generated by an annihilation event 316 and detected by a pair of radiation detectors 320.

The coincidence data is communicated to a sorter 326 where the coincidence events are grouped into projection images, which may be referred to as sinograms. The sorter 326 sorts each sinogram by the angle of each view, which may be measured as the angle, θ, of the line-of-response 318 from a reference direction that lies in the plane of the detector ring assembly 302. For three-dimensional images, the sorter 326 may also sort the sinograms by the tilt of each view. The sorter 326 may also process and sort additional data corresponding to detected photons, including the time at which the photons were detected and their respective energies.

After sorting, the sinograms are provided to the data processing system 306 for processing and image reconstruction. The data processing system 306 may include a data store 328 for storing the raw sinogram data. Before image reconstruction, the sinograms generally undergo preprocessing to correct the sinograms for random and scatter coincidence events, attenuation effects, and other sources of error. The stored sinogram data may thus be processed by a processor 330 located on the data processing system 306, by the operator workstation 308, or by a networked workstation 332.

The operator workstation 308 typically includes a display 334; one or more input devices 336, such as a keyboard and mouse; and a processor 338. The processor 338 may include a commercially available programmable machine running a commercially available operating system. The operator workstation 308 provides the operator interface that enables scan prescriptions to be entered into the PET system 300. In general, the operator workstation 308 may be in communication with a gantry controller 340 to control the positioning of the detector ring assembly 310 with respect to the subject 314 and may also be in communication with the data acquisition system 304 to control operation of the imaging hardware system 302 and data acquisition system 304 itself.

The operator workstation 308 may be connected to the data acquisition system 304 and data processing system 306 via a communication system 342, which may include any suitable network connection, whether wired, wireless, or a combination of both. As an example, the communication system 342 may include both proprietary or dedicated networks, as well as open networks, such as the internet.

The PET system 300 may also include one or more networked workstations 332. By way of example, a networked workstation 332 may include a display 344; one or more input devices 346, such as a keyboard and mouse; and a processor 348. The networked workstation 332 may be located within the same facility as the operator workstation 308, or in a different facility, such as a different healthcare institution or clinic. Like the operator workstation 308, the networked workstation 332 can be programmed to implement the methods and algorithms described here.

The networked workstation 332, whether within the same facility or in a different facility as the operator workstation 308, may gain remote access to the data processing system 306 or data store 328 via the communication system 342. Accordingly, multiple networked workstations 332 may have access to the data processing system 306 and the data store 328. In this manner, sinogram data, reconstructed images, or other data may exchanged between the data processing system 306 or the data store 328 and the networked workstations 332, such that the data or images may be remotely processed by a networked workstation 332. This data may be exchanged in any suitable format, such as in accordance with the transmission control protocol (“TCP”), the internet protocol (“IP”), or other known or suitable protocols.

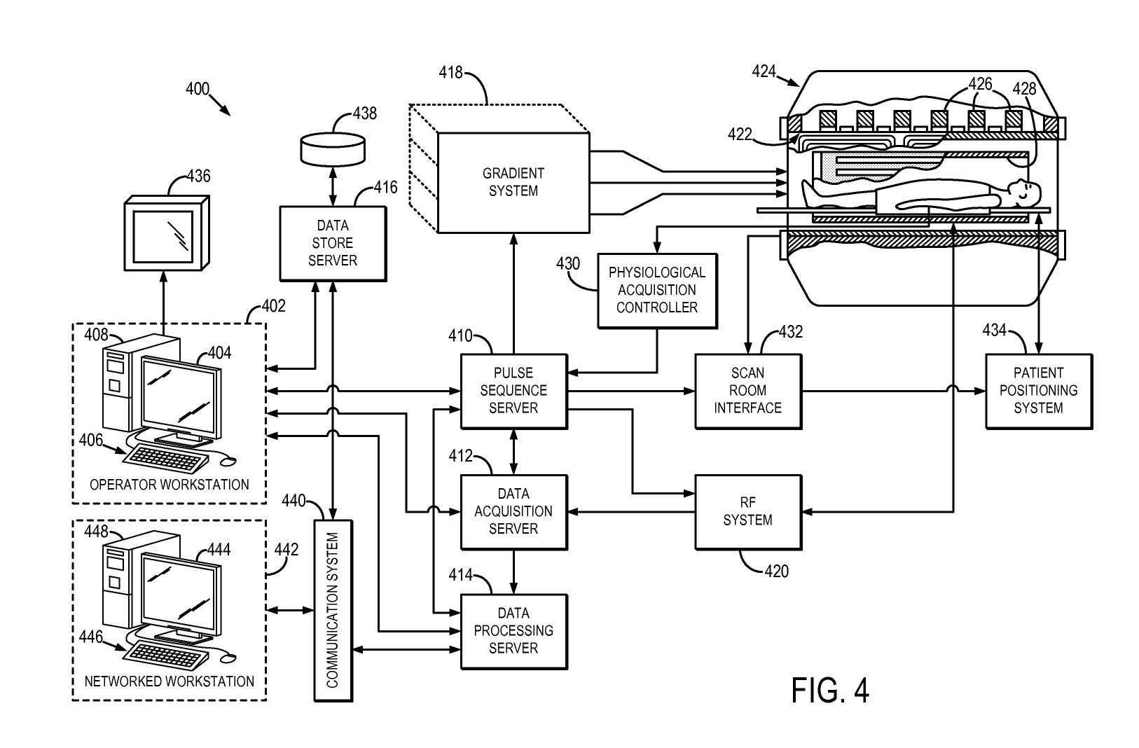

Referring particularly now to FIG. 4, an example of a magnetic resonance imaging (“MRI”) system 400 is illustrated. In some embodiments, the MRI system 400 corresponds to a stand-alone MRI system; however, it will be appreciated by those skilled in the art that the MRI system 400 can also be integrated in a combined imaging system, such as a combined PET and MRI system, such as by integrating a PET system such as the one illustrated above in FIG. 3.

The MRI system 400 includes an operator workstation 402, which will typically include a display 404; one or more input devices 406, such as a keyboard and mouse; and a processor 408. The processor 408 may include a commercially available programmable machine running a commercially available operating system. The operator workstation 402 provides the operator interface that enables scan prescriptions to be entered into the MRI system 400. In general, the operator workstation 402 may be coupled to four servers: a pulse sequence server 410; a data acquisition server 412; a data processing server 414; and a data store server 416. The operator workstation 402 and each server 410, 412, 414, and 416 are connected to communicate with each other. For example, the servers 410, 412, 414, and 416 may be connected via a communication system 440, which may include any suitable network connection, whether wired, wireless, or a combination of both. As an example, the communication system 440 may include both proprietary or dedicated networks, as well as open networks, such as the internet.

The pulse sequence server 410 functions in response to instructions downloaded from the operator workstation 402 to operate a gradient system 418 and a radiofrequency (“RF”) system 420. Gradient waveforms necessary to perform the prescribed scan are produced and applied to the gradient system 418, which excites gradient coils in an assembly 422 to produce the magnetic field gradients Gₓ, G_{y}, and G_{z} used for position encoding magnetic resonance signals. The gradient coil assembly 422 forms part of a magnet assembly 424 that includes a polarizing magnet 426 and a whole-body RF coil 428.

RF waveforms are applied by the RF system 420 to the RF coil 428, or a separate local coil (not shown in FIG. 4), in order to perform the prescribed magnetic resonance pulse sequence. Responsive magnetic resonance signals detected by the RF coil 428, or a separate local coil (not shown in FIG. 4), are received by the RF system 420, where they are amplified, demodulated, filtered, and digitized under direction of commands produced by the pulse sequence server 410. The RF system 420 includes an RF transmitter for producing a wide variety of RF pulses used in MRI pulse sequences. The RF transmitter is responsive to the scan prescription and direction from the pulse sequence server 410 to produce RF pulses of the desired frequency, phase, and pulse amplitude waveform. The generated RF pulses may be applied to the whole-body RF coil 428 or to one or more local coils or coil arrays (not shown in FIG. 4).

The RF system 420 also includes one or more RF receiver channels. Each RF receiver channel includes an RF preamplifier that amplifies the magnetic resonance signal received by the coil 428 to which it is connected, and a detector that detects and digitizes the I and Q quadrature components of the received magnetic resonance signal. The magnitude of the received magnetic resonance signal may, therefore, be determined at any sampled point by the square root of the sum of the squares of the and Q components:

M=\sqrt{I²+Q²} (3);

and the phase of the received magnetic resonance signal may also be determined according to the following relationship:

$$ϕ = {tan}^{- 1} ( \frac{Q}{I} ) . \qquad ( 4 )$$

The pulse sequence server 410 also optionally receives patient data from a physiological acquisition controller 430. By way of example, the physiological acquisition controller 430 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 410 to synchronize, or “gate,” the performance of the scan with the subject's heart beat or respiration.

The pulse sequence server 410 also connects to a scan room interface circuit 432 that receives signals from various sensors associated with the condition of the patient and the magnet system. It is also through the scan room interface circuit 432 that a patient positioning system 434 receives commands to move the patient to desired positions during the scan.

The digitized magnetic resonance signal samples produced by the RF system 420 are received by the data acquisition server 412. The data acquisition server 412 operates in response to instructions downloaded from the operator workstation 402 to receive the real-time magnetic resonance data and provide buffer storage, such that no data is lost by data overrun. In some scans, the data acquisition server 412 does little more than pass the acquired magnetic resonance data to the data processor server 414. However, in scans that require information derived from acquired magnetic resonance data to control the further performance of the scan, the data acquisition server 412 is programmed to produce such information and convey it to the pulse sequence server 410. For example, during prescans, magnetic resonance data is acquired and used to calibrate the pulse sequence performed by the pulse sequence server 410. As another example, navigator signals may be acquired and used to adjust the operating parameters of the RF system 420 or the gradient system 418, or to control the view order in which k-space is sampled. In still another example, the data acquisition server 412 may also be employed to process magnetic resonance signals used to detect the arrival of a contrast agent in a magnetic resonance angiography (“MRA”) scan. By way of example, the data acquisition server 412 acquires magnetic resonance data and processes it in real-time to produce information that is used to control the scan.

The data processing server 414 receives magnetic resonance data from the data acquisition server 412 and processes it in accordance with instructions downloaded from the operator workstation 402. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 414 are conveyed back to the operator workstation 402 where they are stored. Real-time images are stored in a data base memory cache (not shown in FIG. 4), from which they may be output to operator display 402 or a display 436 that is located near the magnet assembly 424 for use by attending physicians. Batch mode images or selected real time images are stored in a host database on disc storage 438. When such images have been reconstructed and transferred to storage, the data processing server 414 notifies the data store server 416 on the operator workstation 402. The operator workstation 402 may be used by an operator to archive the images, produce films, or send the images via a network to other facilities.

The MRI system 400 may also include one or more networked workstations 442. By way of example, a networked workstation 442 may include a display 444; one or more input devices 446, such as a keyboard and mouse; and a processor 448. The networked workstation 442 may be located within the same facility as the operator workstation 402, or in a different facility, such as a different healthcare institution or clinic. Like the operator workstation 402, the networked workstation 442 can be programmed to implement the methods and algorithms described here.

The networked workstation 442, whether within the same facility or in a different facility as the operator workstation 402, may gain remote access to the data processing server 414 or data store server 416 via the communication system 440. Accordingly, multiple networked workstations 442 may have access to the data processing server 414 and the data store server 416. In this manner, magnetic resonance data, reconstructed images, or other data may be exchanged between the data processing server 414 or the data store server 416 and the networked workstations 442, such that the data or images may be remotely processed by a networked workstation 442. This data may be exchanged in any suitable format, such as in accordance with the transmission control protocol (“TCP”), the internet protocol (“IP”), or other known or suitable protocols.

The present invention has been described in terms of one or more preferred embodiments, and it should be appreciated that many equivalents, alternatives, variations, and modifications, aside from those expressly stated, are possible and within the scope of the invention.

We claim:

1. A method for correcting positron emission tomography (PET) data for photon attenuation effects, the steps of the method comprising:
(a) providing to a computer system, a magnetic resonance image that contains data about bone density variations in a subject;
(b) computing linear photon attenuation coefficients with the computer system by mapping signal intensity values in the magnetic resonance image to the linear photon attenuation coefficients;
(c) providing to the computer system for photon attenuation correction, PET data acquired from the subject; and
(d) computing photon attenuation corrected PET data with the computer system by correcting the provided PET data using the linear photon attenuation coefficients.

2. The method of claim 1, wherein the magnetic resonance image is obtained using a water- and fat-suppressed projection imaging (WASPI) pulse sequence.

3. The method of claim 1, wherein computing the linear photon attenuation coefficients includes mapping the signal intensity values in the magnetic resonance image using a calibrated linear mapping function.

4. The method of claim 3, wherein the calibrated linear mapping function is determined by the computer system by comparing the signal intensity values in the magnetic resonance image with calibration data.

5. The method of claim 4, wherein the calibration data are magnetic resonance signal intensity values indicative of a material with a known density.

6. The method of claim 5, wherein the calibration data are determined from a magnetic resonance image depicting a calibration phantom having at least one region composed of the material with the known density.

7. The method of claim 5, wherein the calibration data are determined from at least one region-of-interest in the provided magnetic resonance image, wherein the at least one region-of-interest contains the material with a known density.

8. The method of claim 7, wherein the at least one region-of-interest contains a calibration phantom positioned proximate the subject depicted in the provided magnetic resonance image.

9. The method of claim 7, wherein the material with known density is a tissue contained in the at least one region-of-interest.

10. The method of claim 9, wherein the at least one region-of-interest comprises a first region-of-interest containing a first tissue having a first tissue having a first density and a second region-of-interest containing a second tissue having a second density.

11. The method of claim 10, wherein the first tissue is cortical bone and the second tissue is spongy bone.
